(19) United States
(12) Patent Application Publication
(10) Pub. No.: US 2005/0203493 A1
(43) Pub. Date: Sep. 15, 2005

(54) Method for calculating filter clogging factor and bed-side system

(76) Inventors: Yasuhiro Kuroda, Tokushima-shi, Tokushima (JP); Toshiya Okahisa, Tokushima-shi, Tokushima (JP); Yoshiaki Ohnishi, Tokushima (JP)

Correspondence Address: PHILIPS INTELLECTUAL PROPERTY & STANDARDS, 595 MINER ROAD, CLEVELAND, OH, 44143, US

(21) Appl. No.: 10/516,792

(22) PCT Filed: Jun. 23, 2003
(86) PCT No.: PCT/IB03/02649

§ 371 Date: Dec. 3, 2004

(51) Int. Cl. A61B 18/18

(52) U.S. Cl. 606/5

(57) ABSTRACT

Method for calculating a clogging factor of a filter composed of hollow-fiber membrane, which has a blood inflow portion 32a and a blood outflow portion 32b, by passing a blood, the method including the steps of measuring at least two pressure selected from the group consisting of a pressure (Pa) in said blood inflow portion, a pressure (Pv) in said blood outflow portion, a filtering pressure (Pf1) in said blood inflow portion, and a filtering pressure (Pf2) in said blood outflow portion and calculating a filter clogging factor in vertical direction and/or a filter clogging factor in lateral direction using at least two of the measured pressures, flow rate information, biometric information (viscosity information and so on), and structure information.

### BACKGROUOND OF THE INVENTION

1. Field of the Invention

The present invention relates to a method for calculating filter clogging factor, method and apparatus for monitoring filter clogging, and bed-side system provided with an apparatus for monitoring a filter clogging factor, which are used in the blood purification method.

2. Description of Related Art

A blood purification method can be roughly classified into two types. One is a type that removes substances in the blood through removal to liquid waste (dialysis, filtering) or adsorption into membranes when the blood flows in hollow-fibers of a filter, and hemodialysis, hemofiltering, hemodiafiltering, plasma exchange, double filtering plasmapheresis, plasmapheresis are some examples of this type. The other is a type that removes substances in the blood through adsorption into an adsorbent in a filter when the blood passes through the adsorbent (cloth, bead, etc.), and blood adsorption is an example of this type.

A blood purification method requires a filter for filtering blood. When the purification method is actually applied, several tens of types of filters with different membrane materials, membrane areas and shapes are used at clinical work front depending on the type of the blood purification method applied, clinical conditions of the patient, etc. For example, when a filter with a large membrane area is used, the capacity of removal of substances may be improved, but the amount of blood retained outside the body (in the filter) increases, which increases the possibility of causing a blood pressure drop, and therefore it is essential to select a filter suited to the physical constitution and clinical conditions of the patient. Furthermore, blood purification apparatuses and blood purification circuits of different types (manufacturing companies, model numbers) are used.

Since clogging of filter in this blood purification method may cause problems in terms of safety and economical efficiency, anti-clogging measures by using an anticoagulant or adjusting flow rate, etc., are taken on the medical work front.

Overdosage of this anticoagulant in an attempt to prevent clogging of a filter may not only cause a danger of producing serious hemorrhagic complications (cerebral hemorrhage, etc.) but also raise an economical question because the anticoagulant is expensive. Therefore, it is desirable to discover clogging of a filter in an early stage, adjust the amount of dosage of the anticoagulant appropriately and adjust its flow rate to prevent the progress of clogging. However, it is difficult that a level of filter clogging is monitored accurately.

Filter clogging is currently monitored only based on pressure indices and the degree of clogging is experimentally presumed by observing variations in the pressure indices. However, in the case of monitoring only based on pressure indices, if any one of the types of the filter, blood purification apparatus and blood purification circuit used changes or the flow rate setting changes in the execution of the blood purification method or further the viscosities of the blood and liquid waste change, then the measured pressure changes though the degree of filter clogging remains the same. Therefore, it has been impossible to precisely evaluate a filter clogging situation or make a comparative analysis of the blood purification method adopted under various conditions (using various filters, blood purification apparatuses, blood purification circuits, flow rate settings, viscosities of blood and liquid waste, etc.) based on pressure indices alone.

Furthermore, in a hemodialysis or hemodiafiltering, etc., it is known that mixing of substances contained in a dialyzing fluid with the blood or so-called back-filtration may take place near the blood outflow portion of the filter. This back-filtration has the function of preventing filter clogging. However, if filtering pressure measured at one point is used, it is difficult that filter clogging in filter having back-filtration is monitored accurately.

### SUMMARY OF THE INVENTION

The present invention has been implemented in view of the above-described problems, and it is an object of the present invention to provide a method for calculating a filter clogging factor, method and apparatus for monitoring filter clogging on the basis of the filter clogging factor, and a blood purification apparatus provided with the apparatus for monitoring the filter clogging factor in order to precisely and specifically monitoring filter clogging in blood purification for patients having various conditions of a disease using different filters, blood purification apparatus, or blood purification circuits in several flow rate settings (including back-filtration).

The present invention provides a method for calculating a clogging factor of a filter composed of hollow-fiber membrane, which has a blood inflow portion and a blood outflow portion, for filtering a blood by passing said blood, said method comprising the steps of: measuring at least two pressure selected from the group consisting of a pressure in said blood inflow portion, a pressure in said blood outflow portion, a filtering pressure in said blood inflow portion, and a filtering pressure in said blood outflow portion; and calculating a filter clogging factor indicating the reduction in flowing ease of the blood in said filter and/or a filter clogging factor indicating the reduction in ease of filtering of said filter, by using the measured pressure.

It is also possible to integrate at least two of the flow rate information, measured pressure indices, biometric information (viscosity information) and/or filter structure information, and further obtain a correction coefficient calculated from the pressure indices during priming (operation to connect a circuit and clean the circuit with physiological saline: preparation stage prior to clinical use or after starting blood purification process) and thereby monitor the clogging of a filter irrespective of factors affecting the pressure indices (filter structure, blood purification apparatus, blood purification circuit, flow rate, biometric factor).

According to this method, it is possible to discover filter clogging in an early stage, appropriately adjust the amount of dosage of an anticoagulant without overdosage, change a flow rate setting and thereby prevent the progress of filter clogging. Furthermore, it is also possible to set a flow rate considering back-filtration of each filter by controlling back-filtration.

In the method for calculating a clogging factor of a filter according to the present invention, it is preferable to calculate a filter clogging factor indicating the reduction in flowing ease of the blood in the filter by using a viscosity of blood. This makes it possible to precisely evaluate a level of clogging indicating the reduction in flowing ease of the blood in the filter.

In the method for calculating a clogging factor of a filter according to the present invention, a filter clogging factor indicating the reduction in flowing ease of the blood in said filter is calculated by using structure information and/or flow rate information of the filter. This makes it possible to precisely evaluate a level of clogging indicating the reduction in flowing ease of the blood in the filter.

In the method for calculating a clogging factor of a filter according to the present invention, it is preferable to calculate a filter clogging factor [F(%)], which the reduction in flowing ease of the blood in said filter is represented by the decreasing rate in a cross sectional area inside said hollow-fiber, by using the Equation (1):
F=100{1−[10⁻⁹·K·l·η_{b}·(Q_{b}−Q_{f}/2)/N/ΔP_{b}′/π]^{0.5}/R₀²} Equation (1)
where K represents a correction coefficient (−), η_{b} represents viscosity (Pa·sec) of the blood, Q_{b} represents flow rate (ml/min) of the blood flowing into the filter, Q_{f} represents filtering flow rate (ml/min), N represents the number of hollow-fibers (−), ΔP_{b}′ represents a difference (mmHg) of the pressure between both ends of the hollow-fiber, l represents an effective length (m) of the hollow-fiber, and R₀ represents the radius (m) inside the hollow-fiber that the clogging does not occur.

In the method for calculating a clogging factor of a filter according to the present invention, it is preferable to calculate a filter clogging factor [F(%)], which the reduction in flowing ease of the blood in said filter is represented by the decreasing rate in a cross sectional area inside said hollow-fiber, by using the Equation (2):
F=100{1−[K′·←_{b}(Q_{b}−Q_{f}/2)/ΔP_{b}′]^{0.5}} Equation (2)
where K′ represents a correction coefficient (−), η_{b} represents viscosity (Pa·sec) of the blood, Q_{b} represents flow rate (ml/min) of the blood flowing into the filter, Q_{f} represents filtering flow rate (ml/min), and A P_{b}′ represents a difference (mmHg) of the pressure between both ends of the hollow-fiber.

In the method for calculating a clogging factor of a filter according to the present invention, it is preferable to calculate a filter clogging factor indicating the reduction in flowing ease of the blood in said filter in real-time.

In the method for calculating a clogging factor of a filter according to the present invention, it is preferable to calculate a filter clogging factor indicating the reduction in ease of filtering using the filter by using a viscosity of liquid waste.

In the method for calculating a clogging factor of a filter according to the present invention, a filter clogging factor indicating the reduction in ease of filtering of the filter is calculated by using structure information and/or flow rate information of the filter. This makes it possible to precisely evaluate a level of clogging indicating the reduction in ease of filtering of the filter.

In the method for calculating a clogging factor of a filter according to the present invention, it is preferable to calculate a filter clogging factor [f(%)], which the reduction in ease of filtering of said filter is represented by the decreasing rate in a cross sectional area of pore of said hollow-fiber, by using the Equation (3):
f=100[1−(10⁻⁹·k·τ·ΔX·η_{w}·Q_{f}/r₀²/Aₖ/Aₘ/ΔP_{w}′)^{0.5}] Equation (3)
where k represents a correction coefficient (−), τ represents a rate of curved path, Δ X represents a thickness of a membrane, η_{w} represents a viscosity of liquid waste passing a filter (Pa·sec), Q_{f} represents filtering rate (ml/min), r₀ represents the radius (m) of a hollow-fiber membrane pore that the clogging does not occur, ΔP_{w}′ represents a difference of the pressure between the blood side end and the liquid waste side end in the membrane pore of the filter (mmHg), Aₖ represents a proportion of a cross sectional area of the membrane pore to a unit area of the membrane in the filter, and Aₘ represents an area (m²) of the membrane in the filter.

In the method for calculating a clogging factor of a filter according to the present invention, it is preferable to calculate a filter clogging factor [f(%)], which the reduction in ease of filtering of said filter is represented by the decreasing rate in a cross sectional area of pore of said hollow-fiber, by using the Equation (4):
f=100[1−(k′·η_{w}·Q_{f}/ΔP_{w}′)^{0.5}] Equation (4)
where k′ represents a correction coefficient (−), η_{w} represents a viscosity of liquid waste passing a filter (Pa·sec), Q_{f} represents filtering rate (ml/min), r represents the radius (m) of a hollow-fiber membrane pore that the clogging does not occur, and ΔP_{w}′ represents a difference of the pressure between the blood side end and the liquid waste side end in the membrane pore of the filter (mmHg).

In the method for calculating a clogging factor of a filter according to the present invention, it is preferable to calculate a filter clogging factor indicating the reduction in ease of filtering of the filter in real-time.

In the method for calculating a clogging factor of a filter according to the present invention, it is preferable to calculate a filter clogging factor [S(−)], which the reduction in flowing ease of the blood in the filter is represented by the decreasing rate in a cross sectional area inside said hollow-fiber, by using the Equation (5):
S=[η_{b}·(Q_{b}−Q_{f}/2)·ΔP_{b0}′/η_{b0}/(Q_{b0}−Q_{f0}/2)/ΔP_{b}′]^{0.5} Equation (5)
wherein η_{b} represents viscosity (Pa·sec) of the blood flowing in the hollow-fiber, η_{b0} represents viscosity (Pa·sec) of the priming liquid in the priming, Q_{b} represents flow rate (ml/min) of the blood flowing into the filter, Q_{b0} represents flow rate (ml/min) of the priming liquid flowing into the filter in the priming, Q_{f} represents filtering flow rate (ml/min), Q_{f0} represents filtering flow rate (ml/min) of the priming liquid flowing into the filter, ΔP_{b}′ represents a difference (mmHg) (Pa−Pv) of the pressure between both ends of the hollow-fiber, and ΔP_{b0}′ represents a difference (mmHg) of the pressure between both ends of the hollow-fiber in the priming. Here, values (viscosity of the blood flowing in the hollow-fiber, flow rate of the blood flowing into the filter, filtering flow rate, a difference of the pressure between both ends of the hollow-fiber), that are obtained after starting blood purification process, may be used as η_{b0}, Q_{b0}, Q_{f0} and ΔP_{b0}′.

In the method for calculating a clogging factor of a filter according to the present invention, it is preferable to calculate a filter clogging factor [s(−)], which the reduction in ease of filtering of the filter is represented by the decreasing rate in a cross sectional area of membrane pore of said hollow-fiber, by using the Equation (6):
S=(η_{w}·Q_{f}·ΔP_{w0}′/η_{w0}/Q_{f0}/ΔP_{w}′)^{0.5} Equation (6)
wherein η_{w} represents viscosity (Pa·sec) of the liquid waste, ηw₀ represents viscosity (Pa·sec) of the liquid waste in the priming, Q_{f} represents filtering flow rate (ml/min), Q_{f0} represents filtering flow rate (ml/min) of the priming liquid, ΔP_{w}′ represents a difference (mmHg) of the pressure between blood side end and liquid waste side end of the hollow-fiber membrane pore, ΔP_{w0}′ represents a difference (mmHg) of the pressure between blood side end and liquid waste side end of the hollow-fiber membrane pore in the priming, and s represents a ratio of cross sectional areas in the hollow-fiber membrane pore of the filter. Here, values (viscosity of the liquid waste, filtering flow rate, a difference of the pressure between blood side end and liquid waste side end of the hollow-fiber membrane pore), that are obtained after starting blood purification process, may be used as η_{w0}, Q_{f0} and ΔP_{w0}′.

This makes it possible to eliminate the influences of errors included in the correction coefficient in the calculation equation of the filter clogging factor and monitor the filter clogging more precisely irrespective of factors affecting the pressure indices (filter structure, blood purification apparatus, blood purification circuit, flow rate, biometric factor).

In the method for calculating a clogging factor of a filter according to the present invention, it is preferable to use an average of ΔPW′ in the blood inflow portion and ΔPW′ in the blood outflow portion of the filter as A PW′.

The present invention provides a method for monitoring a clogging of a filter comprising the steps of calculating a clogging factor of a filter by using the above-described method for calculating a clogging factor of a filter and monitoring a clogging of a filter on the basis of the clogging factor of a filter.

Furthermore, the present invention provides an apparatus for monitoring a clogging of a filter comprising means for calculating a clogging factor of a filter by using the above-described method for calculating a clogging factor of a filter and means for monitoring a clogging of a filter on the basis of the clogging factor of a filter.

Furthermore, the present invention provides a bed-side system comprising the above-described apparatus for monitoring a clogging of a filter.

### BRIEF DESCRIPTION OF THE DRAWINGS

FIG. 1a is a view showing a filter used in the blood purification;

FIG. 1b is a view showing a hollow-fiber in a filter;

FIG. 2 is a view to explain a clogging in vertical direction and in lateral direction;

FIG. 3 is a view to explain a portion of measuring the pressure that is used in a method according to the present invention;

FIG. 4 is a view to explain the pressure that is used in a method according to the present invention;

FIG. 5 is a view to explain the back-filtration;

FIG. 6 is a view showing an arrangement of a bed-side system which implements a method according to the present invention;

FIG. 7 illustrates a variation of the clogging factor (F) in the vertical direction when sustained blood filtering was performed;

FIG. 8 illustrates a variation of the clogging factor (f) in the horizontal direction when sustained blood filtering was performed;

FIG. 9 illustrates a variation of a pressure index Pa−Pv and a variation of the clogging factor (F) in the vertical direction caused by a variation in the blood flow rate;

FIG. 10 shows a simulation curve indicating a relationship between the pressure index Pa−Pv and clogging factor F (%) in the vertical direction; and

FIG. 11 illustrates a variation of the clogging factor (s) in the horizontal direction when sustained blood filtering was performed.

### DESCRIPTION OF THE PREFERRED EMBODIMENT(S)

The present inventors have noticed the fact that there are two modes of filter clogging; (vertical) clogging indicating the reduction in flowing ease of the blood in the filter and (lateral) clogging indicating the reduction in ease of filtering using the filter, and have come up with the present invention by discovering that it is possible to accurately monitor filter clogging by calculating a filter clogging factor on the basis of a relationship between clogging in each mode of clogging and pressure.

That is, a subject matter of the present invention is to measure at least two pressures selected from the group consisting of a pressure in the blood inflow portion, a pressure in the blood outflow portion, a filtering pressure in the blood inflow portion, and a filtering pressure in the blood outflow portion of the filter and calculate filter clogging factors in vertical direction and lateral direction using the measured pressures. This makes it possible to discover filter clogging in an early stage, appropriately adjust the amount of dosage of an anticoagulant without overdosage, change a setting of flow rate of blood and thereby prevent the progress of filter clogging.

Furthermore, it is also possible to integrate at least two the flow rate information, measured pressure indices, biometric information (viscosity information and so on) and/or filter structure information, and further obtain a correction coefficient calculated from the pressure indices during priming (operation to connect a circuit and clean the circuit with physiological saline: preparation stage prior to clinical use) and thereby monitor the clogging of a filter irrespective of factors affecting the pressure indices (filter structure, blood purification apparatus, blood purification circuit, flow rate, biometric factor).

With reference now to the attached drawings, embodiments of the present invention will be explained in detail below.

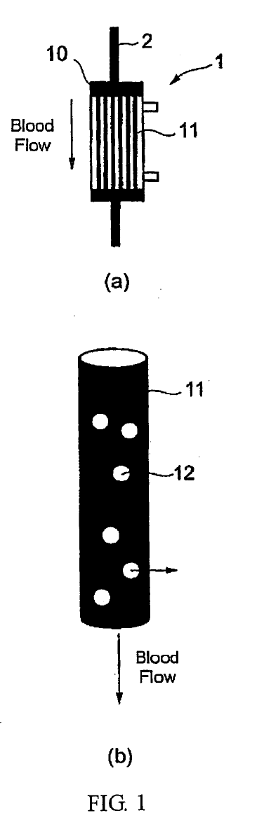

First, a filter clogging mode will be explained using FIG. 1. A filter 1 used for blood purification is composed of on the order of several thousand to 10,000 hollow-fibers 11 placed in a housing 10 as shown in FIG. 1(a), each hollow-fiber 11 having an effective length of approximately 150 to 250 mm and an inside diameter of approximately 200 μm in a humid condition. This filter 1 is connected to a circulation path 2 for circulating bodily fluids such as blood. Furthermore as shown in FIG. 1(b), many membrane pores 12 of several hundred nanometers in diameter are formed on the side of the hollow-fibers 11. Clogging in such hollow-fibers during blood purification of the hollow-fibers can be roughly classified into two types of clogging; clogging inside of the hollow-fibers (clogging indicating the reduction in flowing ease of the blood: vertical clogging) and clogging of membrane pores of the hollow-fiber membranes (clogging indicating the reduction in ease of filtering: lateral clogging).

Clogging in the hollow-fibers during blood purification process may be caused by 1) adsorption of protein onto the membrane surface or into the membrane, 2) adhesion or invagination of fibrin onto the membrane surface, 3) adhesion or invagination of blood platelets onto the membrane surface, 4) adhesion of white blood cells onto the membrane surface, 5) adhesion of red blood cells onto the membrane surface, and 6) adhesion of medicine onto the membrane surface or into the membrane, etc.

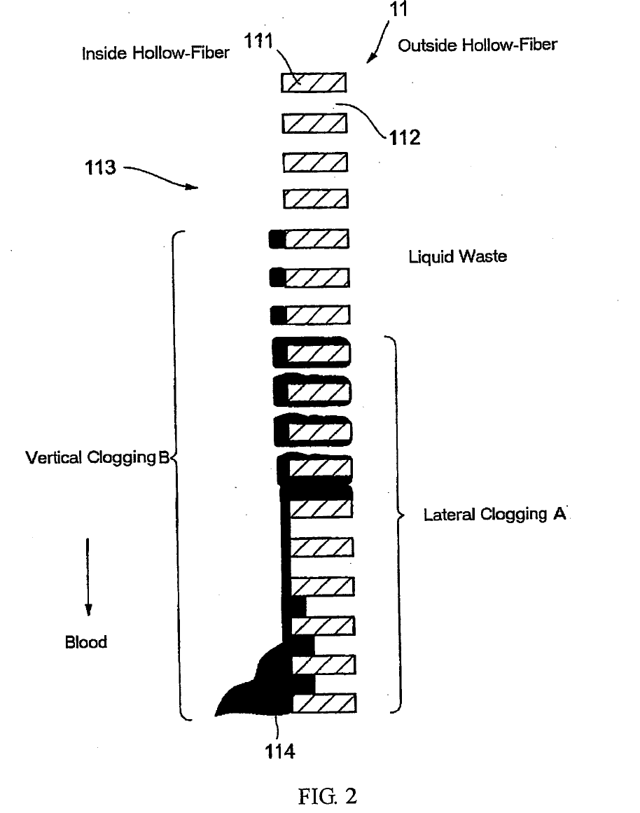

As shown in FIG. 2, adhesion of substances onto the membrane surface of the hollow-fibers causes not only clogging (lateral clogging) A of membrane pores 112 of the hollow-fiber membrane 111 of the hollow-fiber 11 but also clogging inside of the hollow-fiber 113 (vertical clogging) B due to a narrowing of the inside 113 of the hollow-fiber simultaneously. The clogging inside 113 of the hollow-fiber (vertical clogging) B is only caused by adhesion of substances (e.g., protein, fibrin, blood platelets, blood cells, medicine) 114 onto the surface of the hollow-fiber membrane 111, while clogging (lateral clogging) A of membrane pores 112 of the hollow-fiber membrane 111 is caused by adhesion of the substances 114 not only onto the surface of the hollow-fiber membranes 111 but also into the membrane pores 112 of the hollow-fiber membrane 111. Furthermore, liquid waste (filtrate, dialyzing fluid) exists outside the hollow-fibers.

Clogging inside the hollow-fiber (vertical clogging) B causes the reduction in the blood flow rate in the hollow-fiber where the clogging occurs and the reduction in the ability to remove substances by means of diffusion. The reduction in the blood flow rate facilitates adhesion of substances onto the membrane and makes clogging more likely to occur. Complete clogging of the inside of the hollow-fibers not only makes it impossible to remove substances of the hollow-fibers at the outlet from the clogged portion but also allows to the blood to remain in the filter (residual blood) at the end of blood purification, leading to a blood loss of the patient.

Clogging (lateral clogging) A of membrane pores of the hollow-fiber membrane involves a danger of causing the reduction in the ability to remove substances (clearance), suctions blood cells that have a larger diameter than membrane pores and do not pass through the membrane pores by a strong negative pressure and has a possibility of causing destruction of blood cells (hemolysis, etc.). The ease of filtering refers to ease of filtrate that passes through the filter with which the filtrate passes to the liquid waste side, and this reduces when the clogging factor f of a filter increases and when the clogging factor s of a filter decrease.

Portions of clogging of these membranes and the degree of clogging are determined by 1) conditions for executing blood purification process such as type of a filter, flow rate setting, type and amount of dosage of a coagulant, type of substitution liquid and dialyzing fluid, 2) clinical condition of the patient, 3) medical treatment conditions such as blood transfusion, medicine, medical treatment, etc. Here, adhesion of substances onto the surface of the hollow-fiber membrane is related to clogging in vertical direction and clogging in lateral direction, while adhesion of substances into membrane pores of the hollow-fiber membrane is related to clogging in lateral direction.

As a method for detecting a level of clogging of a filter, there is a method whereby a pressure in a filter and/or a blood purification circuit is measured and a clogging factor of the filter is calculated on the basis of information on the pressure. This embodiment uses a pressure measured in a drip-chamber in the blood inflow portion located between a blood roller pump and the filter (blood inflow portion pressure (arterial pressure: Pa)), a pressure measured in a drip-chamber in the blood outflow portion located after the filter (blood outflow portion pressure (venous pressure: Pv)), a pressure measured outside the hollow-fiber on the blood inflow portion side of the filter (filtering pressure in the blood inflow portion: Pf1) and a pressure measured outside the hollow-fiber on the blood outflow portion side of the filter (filtering pressure in the blood outflow portion: Pf2) and calculates filter clogging factors in vertical direction and in lateral direction using other information ((flow rate information, biometric information (viscosity information and the like), filter structure information (membrane material, diameter of hollow-fiber, effective length of hollow-fiber, membrane area, membrane thickness, rate of hollow area, rate of curved path, diameter of membrane pore)).

As will be described later, the filter clogging factor according to the present invention is calculated based on the Hagen-Poiseuille law. However, filter structure information substituted into the Hagen-Poiseuille law for calculating the filter clogging factor is a general value. There is a difference between the general value and a value used actually. Further, it is necessary to calculate a filter clogging factor with correcting errors during pressure measurement (pressure loss of blood purification circuit) or errors of biometric information (values calculated by an approximate expression).

Thus, in the present invention, for evaluating a level of filter clogging more accurately, a filter clogging factor (F, f) is calculated by equations including correction coefficients K, K, k and k′, and a filter clogging factor (S, s) is calculated.

When a clogging factor in vertical direction is calculated, the clogging factor is calculated using at least two of a correction coefficient, a flow rate of the blood, pressure information (a difference of the pressure between the blood inflow portion pressure and the blood outflow portion pressure and so on), biometric information (viscosity information), and filter structure information (the number of hollow-fibers, radius of the hollow-fiber that the clogging does not occur, and so on). In this case, the blood viscosity can be calculated using any one of the following methods:
1. Approximation using hematoclit value continuously measured by a clitline monitor or actually measured hematoclit value (Ht) and actually measured blood protein level (TP).
2. Approximation using only hematoclit value continuously measured by a clit line monitor or actually measured hematoclit value (Ht).
3. Actual measurement by viscometer

The difference of the pressure Pa−Pv can be calculated continuously from the actually measured values of Pa and Pv and the flow rate of the blood is a set value, and therefore by approximating a blood viscosity using hematoclit value continuously measured by a clitline monitor or only actually measured hematoclit value, it is possible to calculate a filter clogging factor in vertical direction in real-time.

When a clogging factor in lateral direction is calculated, the clogging factor is calculated using at least two of a correction coefficient, a flow rate of filtering, pressure information (TMP (transmembrane pressure), which is a difference of the pressure between membranes representing a pressure that contributes to filtering, biometric information (viscosity of liquid waste and so on) and filter structure information (radius of the hollow-fiber that the clogging does not occur, rate of curved path, membrane thickness and so on). In this case, the viscosity of liquid waste can be obtained by an actual measurement. The TMP can be calculated using any one of the following methods:
1. Calculation by obtaining blood inflow portion pressure Pa, blood outflow portion pressure Pv, filtering pressure at the blood inflow portion Pf1 and filtering pressure at the blood outflow portion Pf2 continuously through actual measurements and using the actually measured values.
2. Calculation using the actually measured values of Pa, Pv, Pf1 and Pf2, blood colloidal osmotic pressure actually measured using a colloidal osmotic pressure gauge and Staverman's coefficient of restitution.
3. Calculation using the actually measured values of Pa, Pv, Pf1 and Pf2, blood colloidal osmotic pressure approximated by Alb/Glb obtained through a clinical examination and Staverman's coefficient of restitution.
4. Calculation using the actually measured values of Pa, Pv, Pf1 and Pf2, blood colloidal osmotic pressure actually measured using a colloidal osmotic pressure gauge and colloidal osmotic pressure of liquid waste.

Thus, by combining pressure information and colloidal osmotic pressure information, it is possible to grasp back-filtration of hemodialysis and grasp clogging in lateral direction more precisely.

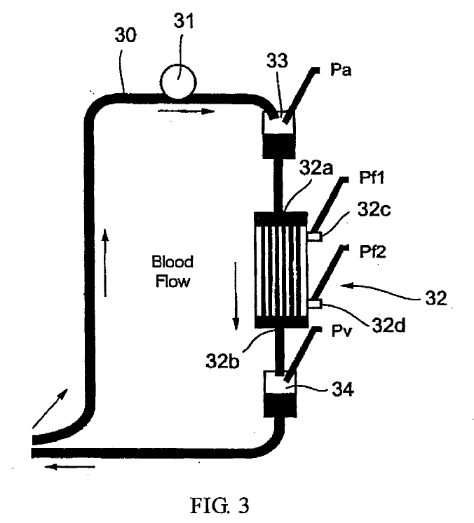

Then, portions of measuring Pa, Pv, Pf1 and Pf2 will be explained. Pa, Pv, Pf1 and Pf2 will be measured at the portions shown in FIG. 3. In FIG. 3, a roller pump 31 is connected to a circulation path 30 along which the blood flows. This roller pump 31 circulates blood (bodily fluid) through the circulation path 30 outside the body. The circulation path 30 is provided with a filter 32 that filters the blood. This filter 32 is provided with a blood inflow portion 32a and blood outflow portion 32b, also provided with a coupler 32c of the blood inflow portion and a coupler 32d of the blood outflow portion which serve as the outlet and inlet of a dialyzing liquid and liquid waste. The couplers 32c and 32d are connected to their respective tubes (not shown) and the pressures in those tubes become the filtering pressure of the blood inflow portion (Pf1) and the filtering pressure of the blood outflow portion (Pf2) respectively.

Furthermore, before the filter 32 on the circulation path 30, a blood inflow portion drip-chamber 33 is provided. On the other hand, after the filter 32 on the circulation path 30, a blood outflow portion drip-chamber 34 is provided. According to this embodiment, the pressures Pa and Pv of the blood inflow portion 32a and blood outflow portion 32b of the filter 32 are measured at the blood inflow portion drip-chamber 33 and blood outflow portion drip-chamber 34. However, if the pressures in the blood inflow portion 32a and blood outflow portion 32b of the filter 32 can be measured, the Pa and Pv can also be measured at any portions other than the blood inflow portion drip-chamber 33 and blood outflow portion drip-chamber 34.

In such a configuration, a blood inflow portion pressure (arterial pressure: Pa) is measured at the blood inflow portion drip-chamber 33 and a blood outflow portion pressure (venous pressure: Pv) is measured at the blood outflow portion drip-chamber 34, a filtering pressure (Pf1) of the blood inflow portion is measured at the tube connected to the coupler 32c of the blood inflow portion and a filtering pressure (Pf2) of the blood outflow portion is measured at the tube connected to the coupler 32d of the blood outflow portion. By the way, the methods for measuring pressures at the respective portions are the same as the method for measuring pressures during normal detection of filter clogging.

Then, a filter clogging factor will be calculated using at least two of the pressure information, flow rate information, biometric information (viscosity information, osmotic pressure information and so on) and structure information measured as described above. The hematoclit value that defines a blood viscosity can be collected continuously using a continuous hematoclit monitor. The continuous hematoclit monitor is described in the Japanese Patent Application No. 2000-397609, the content of which is also included herein. Thus, calculating a filter clogging factor by combining at least two of the pressure information, flow rate information (blood flow, filtering), biometric information (viscosity information, osmotic pressure information and so on) and filter structure information will make it possible to grasp clogging more precisely.

(Calculation of Filter Clogging Factor [F(%)] in Vertical Direction)

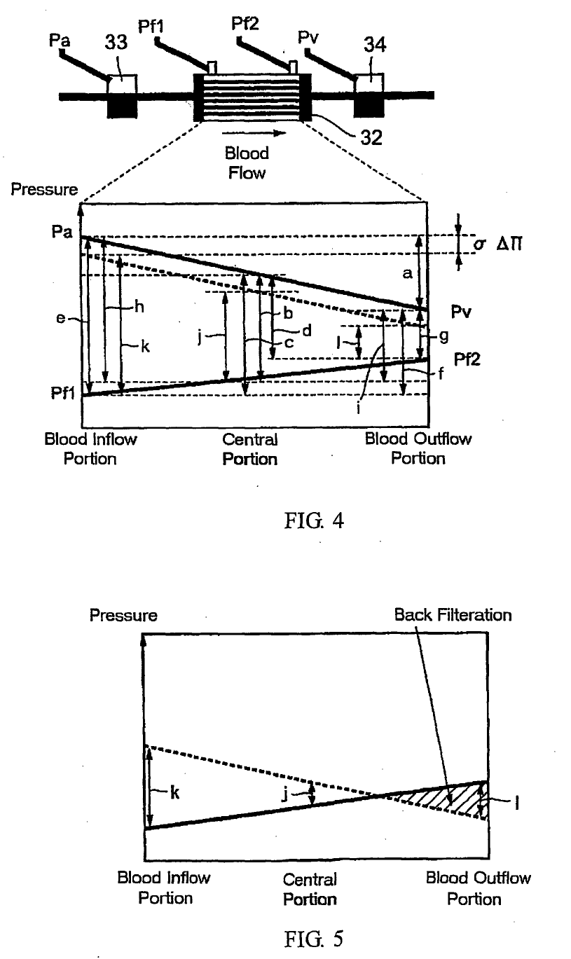

A difference Pa−Pv (a in FIG. 4) of the pressure between the blood inflow portion pressure (arterial pressure: Pa) and blood outflow pressure (venous pressure: Pv) is one of factors expressing clogging (clogging in vertical direction) of the hollow-fiber of a filter. According to a labyrinthine membrane pore theory, which is well known in the art, when a fluid flows in a laminar flow in the hollow-fibers of a filter, Pa, Pv is defined by blood flow rate Q_{b} and blood viscosity η_{b} according to Hagen-Poiseuille law. Thus, it is necessary to measure blood viscosity η_{b} in order to evaluate clogging of the hollow-fibers of the filter (clogging in vertical direction) from Pa, Pv.

A viscosity of blood can be calculated approximately from a hematoclit value of the blood and/or blood protein level. A hematoclit value can be measured by a blood test, but when blood purification is performed, the hematoclit value changes together with water elimination and dosage of a substitution liquid. However, when a blood purification treatment is in progress, it is possible to collect information on a hematoclit value noninvasively, in real-time, continuously and automatically through a continuous hematoclit monitor. Therefore, the information on a viscosity of blood obtained through such an approximate calculation may be used to calculate a filter clogging factor.

According to a labyrinthine membrane pore theory, which is well known in the art, when a fluid flows in a laminar flow in the hollow-fiber of a filter, the Hagen-Poiseuille law is held as shown in the following equation (Equation (7)).
Q=π·R⁴·ΔP_{b}/8η_{b}/l Equation (7)
A=π·R² Equation (8)
Q=(Q_{b}−Q_{f}/2)/N/(6×10⁷) Equation (9)
ΔP_{b}=133.3·ΔP_{b}′ Equation (10)
where the respective parameters represent the following:
Q: Flow rate of blood passing through hollow-fibers (m³/sec)
Q_{b}: Flow rate of blood flowing into the filter (ml/min)
Q_{f}: Filtering flow rate (ml/min)
R: Radius of inside of hollow-fiber (m)
N: Number of hollow-fibers (−)
l: Effective length of hollow-fiber (m)
ΔP_{b}: Difference of pressure between both ends of hollow-fiber (corresponds to Pa−Pv in this case) (Pa)
ΔP_{b}′: Difference of pressure between both ends of hollow-fiber (corresponds to
Pa−Pv in this case) (mmHg)
η_{b}: Viscosity of blood passing through hollow-fiber (Pa·sec)
A: Cross sectional area of inside of hollow-fiber (m²)

From Equations (7) to (10), cross sectional area A (m²) of inside of hollow-fiber is calculated by Equation (11):
A=[10⁻⁹·π·l·η_{b}·(Q_{b}−Q_{f}/2)/N/ΔP_{b}′]^{0.5} Equation (11)
From Equation (11), cross sectional area A₀ (m²) of inside of hollow-fiber in filter without clogging is calculated by Equation (12):
A=[10⁻⁹·π·l ·η_{b0}·(Q_{b0}−Q_{f0}/2)/N/ΔP_{b0}′]^{0.5} Equation (12)
where the respective parameters represent the following:
η_{b0}: viscosity of the priming liquid (Pa·sec)
Q_{b0}: flow rate of the priming liquid flowing into the filter in the priming (ml/min)
Q_{f0}: filtering flow rate of the priming liquid (ml/min)
N: Number of hollow-fibers (−)
l: Effective length of hollow-fiber (m)
ΔP_{b0}′: a difference of the pressure between both ends of the hollow-fiber in the priming (mmHg)
A₀: Cross sectional area of inside of hollow-fiber without clogging (m²)

Cross sectional area A₀ (m²) of inside of hollow-fiber in filter without clogging is also calculated by Equation (13):
A₀′=π·R₀² Equation (13)
where R₀ represents radius of inside of hollow-fiber without clogging.

Although A₀ (m²) obtained by Equation (12) and A₀′ (m²) obtained by Equation (13) should be same in theory, A₀ (m²) and A₀′ (m²) are not same in actual due to errors between general filter structure information and filter structure information used actually, errors in measuring the pressure (pressure loss of blood purification circuit and so on), and errors of biometric information (obtained by approximate equation). Therefore, it is necessary to set a correction coefficient K(−) indicating Equation (14):
A₀′=K^{0.5}·A₀ Equation (14)
A filter clogging factor F(%), which the reduction in flowing ease of the blood in the filter is represented by the decreasing rate in a cross sectional area inside said hollow-fiber can be calculated by Equation (15):
F=100·(1−A/A₀) Equation (15)
From Equations (14) and (15), a filter clogging factor F(%), which the reduction in flowing ease of the blood in the filter is represented by the decreasing rate in a cross sectional area inside said hollow-fiber can be calculated by Equation (16):
F=100−(1−K^{0.5}A/A₀′) Equation (16)
From Equations (11), (13) and (16), a filter clogging factor F(%), which the reduction in flowing ease of the blood in the filter is represented by the decreasing rate in a cross sectional area inside said hollow-fiber can be calculated by Equation (1):
F=100{1−[10⁻⁹·K·η_{b}·(Q_{b}−Q_{f}/2)/N/ΔP_{b}′/π]^{0.5}/R²} Equation (1)
From Equations (12) to (14), a correction coefficient K(−) in Equation (1) can be calculated by Equation (17):
K=10⁹·π·R₀⁴·N·ΔP_{b0}′/l/η_{b0}/(Q_{b0}−Q_{f0}/2) Equation (17)
where the respective parameters represent the following:
Q_{b0}: a flow rate of the priming liquid that flows through the hollow-fiber in the priming (ml/min)
Q_{f0}: a filtering flow rate in the priming (ml/min)
R₀: a radius of the hollow-fiber without clogging (m)
N: the number of hollow-fibers (−)
l: the effective length of the hollow-fiber (m)
ΔP_{b0}′: a pressure difference between both ends of the hollow-fiber in the priming (mmHg)

η_{b0}: a viscosity of the priming liquid (Pa·sec).

Equation (1) includes a parameter of filter structure information (a radius of the hollow-fiber without clogging, the number of hollow-fibers and the effective length of the hollow-fiber). Thus, it is impossible to calculate a filter clogging factor using Equation (1), if filter structure information is not given. Therefore, if filter structure information is not given, a filter clogging factor F(%), which the reduction in flowing ease of the blood in the filter is represented by the decreasing rate in a cross sectional area inside said hollow-fiber can be calculated by Equation (2) which does not include a parameter of filter structure information, by setting a correction coefficient K′(−) obtained by Equation (18):
K′=10⁻⁹·K·l/N/ΔP_{b0}′/π/R₀⁴ Equation (18)
F=100{1−[K′·η_{b0}·(Q_{b0}−Q_{f0}/2)/ΔP_{b0}]^{0.5}} Equation (2)
From Equations (17) and (18), a correction coefficient K′(−) in Equation (2) can be calculated by Equation (19):
K′=ΔP_{b0}′/η_{b0}/(Q_{b0}−Q_{f0}/2) Equation (19)
where the respective parameters represent the following:
Q_{b0}: a flow rate of the priming liquid that flows through the hollow-fiber in the priming (ml/min)
Q_{f0}: a filtering flow rate in the priming (ml/min)
ΔP_{b0}′: a pressure difference between both ends of the hollow-fiber in the priming (mmHg).

Furthermore, the viscosity (η_{b}Pa·sec)) of the blood that passes through the hollow-fiber can be calculated approximately by using the following Equation (20).
lnη_{b}=10⁻²·Ht·(−0.23TP+3.675)+(0.059TP−0.354) Equation (20)
where the symbols of the respective parameters represent the following:
Ht: Hematoclit value (%)
TP: Blood protein level (g/di)

From the above Equations (1) to (20), the clogging factor F (%) of a filter in vertical direction can be calculated by using the following Equation (1′). Here, the clogging factor F (%) of a filter in vertical direction means a proportion of a cross sectional area of the hollow-fibers in which the blood flows to a cross sectional area inside the hollow-fibers of a filter without clogging.
F=100[1−{10⁻⁹·K·l·exp[10⁻²·Ht(−0.23TP+3.675)+(0.059TP−0.354)]·(Q_{b}−Q_{f}/2)/N/ΔP_{b}′/π}^{0.5}/R₀²] Equation (1′)
where K represents a correction coefficient(−) calculated by Equation (17).

From the above Equations (2) to (20), the clogging factor F (%) of a filter in vertical direction can be calculated by using the following Equation (2′):
F=100[1−{K′·exp[10⁻²·Ht(−0.23TP+3.675)+(0.059TP−0.354)]·(Q_{b}−Q_{f}/2)/N/ΔP_{b}′/π}^{0.5}/R₀²] Equation (2′)
where K′ represents a correction coefficient (−) calculated by Equation (19).

Furthermore, the rate AF (%/min) of change per unit time of clogging factor F in vertical direction can be calculated by using the following Equation (21):
ΔF=dF/dt Equation (21)
where t represents time (min).
(Calculation of Filter Clogging Factor [S(−)] in Vertical Direction)

From the above Equations (11) and (12), a filter clogging factor [S(−)](S=A/A₀) which the reduction in flowing ease of the blood in the filter is represented by a ratio of a cross sectional area inside the hollow-fiber, is obtained by Equation (5).
S=[η_{b}·(Q_{b}−Q_{f}/2)ΔP_{b0}′/η_{b0}/(Q_{b0}−Q_{f0}/2)/ΔP_{b}′]^{0.5} Equation (5)
where the respective parameters represent the following:
ηb: viscosity of the blood flowing in the hollow-fiber (Pa·sec)
ηb₀: viscosity of the priming liquid in the priming (Pa·sec)
Q_{b}: flow rate of the blood flowing into the filter (ml/min)
Q_{b0}: flow rate of the priming liquid flowing into the filter in the priming (ml/min)
Q_{f}: filtering flow rate (m/min)
Q_{f0}: filtering flow rate in the priming (ml/min)
ΔP_{b}′: a difference of the pressure between both ends of the hollow-fiber (mmHg) (Pa−Pv)
ΔP_{b0}′: a difference of the pressure between both ends of the hollow-fiber in the priming (mmHg).

Thus, calculated is a filter clogging factor [S(−)](S=A/A₀) which the reduction in flowing ease of the blood in the filter is represented by a ratio of a cross sectional area inside the hollow-fiber using flow rate information, measured pressure indices and biometric information (viscosity information). This makes it possible to eliminate the influences of errors included in the correction coefficient (K,K′) in the calculation equation of the filter clogging factor and monitor the filter clogging more precisely irrespective of factors affecting the pressure indices (filter structure, blood purification apparatus, blood purification circuit, flow rate, biometric factor).

In this embodiment, parameters R₀, N and 1 are defined by the type of the filter and the respective parameters are collected as follows:
R₀, N, l: Manually input
Q_{b}: Measured by the blood purification apparatus and automatically input continuously in real-time.
ΔP_{b}′ (=Pa−Pv): Measured by the blood purification apparatus or pressure information collection apparatus and automatically input continuously in real-time.
Ht: Measured by the continuous hematoclit monitor and automatically input continuously in real-time.
TP: Blood protein level (g/dl) (measured several times a day by a blood test and values are manually input)

However, the input method is not limited to the above-described method.

(Calculation of Filter Clogging Factor [f(%)] in Lateral Direction)

According to a labyrinthine membrane pore theory, which is well known in the art, when a fluid flows in a laminar flow through membrane pores in the hollow-fibers of a filter, the Hagen-Poiseuille law is held as shown in the following equation (Equation (22)):
Q=π·r⁴·ΔP_{w}/8η_{w}/l Equation (22)
A=τr² Equation (23)
l=τ·ΔX Equation (24)
Q=Q_{f}/(6×10⁷·Aₖ·Aₘ·τr²) Equation (25)
ΔP_{w}=133.3·ΔP_{w}′ Equation (26)
where the respective parameters represent the following:
Q: Flow rate of blood passing through membrane pore (m³/min)
Q_{f}: Filtering rate (ml/min)
r: Radius of hollow-fiber (m)
l: Effective length of hollow-fiber (m)
τ: Rate of curved path (m)
ΔX: Thickness of membrane (m)
ΔP_{w}: Difference of pressure between blood side end and liquid waste side end of membrane pore of filter (mmHg)
ΔP_{w}′: Difference of pressure between blood side end and liquid waste side end of membrane pore of filter (mmHg)
η_{w}: Viscosity of blood passing through membrane pore (Pa sec)
A: Cross sectional area of membrane pore of hollow-fiber (m²)
A₀: Cross sectional area of membrane pore of hollow-fiber that the clogging does not occur (m²)
Aₖ: Proportion of cross sectional area of membrane pore to unit area of membrane (−)
Aₘ: Area of membrane (m²).

ΔP_{w}′ is also a pressure actually contributing to filtering at the center of the filter (effective filtering pressure) and a TMP (transmembrane pressure) calculated by Equation (27) that takes into account an osmotic pressure (σΔΠ) by membrane impermeable substances that exist on the blood side. TMP is a difference of the pressure between membranes indicating a pressure that contributes to filtering. This TMP increases as clogging of a filter advances, and therefore TMP not only represents a pressure that contributes to filtering but is also used as a factor for evaluating clogging of a filter.
TMP=(Pa+Pv)/2−(Pf1+Pf2)/2−σΔΠ Equation (27)
AΠ: Colloidal osmotic pressure by protein (mmHg)
σ: Staverman's coefficient of restitution (proportion of solute that cannot permeate membrane)(−)

From Equations (22) to (26), cross sectional area a (m²) of membrane pore is calculated by Equation (28):
a=[10⁻⁹π²·r²τ·ΔX·η_{w}·Q_{f}/Aₖ/Aₘ/ΔP_{w}′,]^{0.5} Equation (28)
From Equation (28), cross sectional area a₀ (m²) of membrane pore without clogging is calculated by Equation (29):
a₀=[10⁻⁹·π²r₀²τ·ΔX·η_{w0}·Q_{f0}/Aₖ/AₘΔP_{w0}′]^{0.5} Equation (29)
where the respective parameters represent the following:
Q_{f0}: filtering flow rate in the priming (ml/min)
r₀: the radius of the hollow-fiber without clogging (m)
τ: a rate of curved path (−)
ΔX: membrane thickness (m)
ΔP_{w0}′: a difference of the pressure between blood side end and liquid waste side end of the hollow-fiber membrane pore in the priming (mmHg)
η_{w0}: the viscosity of the priming liquid (Pa−sec)
a₀: cross sectional area of membrane pore without clogging (m²)
Aₖ: the ratio of the cross sectional area of a membrane pore to a unit area (−)
Aₘ: membrane area (m²)

Cross sectional area a₀ (m²) of inside of hollow-fiber in filter without clogging is also calculated by Equation (30):
a₀′=πr₀² Equation (30)
where r₀ represents radius of membrane pore without clogging.

Although a₀ (m²) obtained by Equation (29) and a₀′(m²) obtained by Equation (30) should be same in theory, a₀ (m²) and a₀′ (m²) are not same in actual due to errors between general filter structure information and filter structure information used actually and errors in measuring the pressure (pressure loss of blood purification circuit and so on). Therefore, it is necessary to set a correction coefficient k(−) indicating Equation (31):
a₀′=k^{0.5}·a₀ Equation (31)

A filter clogging factor f(%), which the reduction in ease of filtering of the filter is represented by the decreasing rate in a cross sectional area of membrane pore can be calculated by Equation (32):
f=100·(1−a/a₀) Equation (32)

From Equations (31) and (32), a filter clogging factor F(%), which the reduction in ease of filtering of the filter is represented by the decreasing rate in a cross sectional area of membrane pore can be calculated by Equation (33):
f=100·(1−k^{0.5}·a/a₀′) Equation (33)

From Equations (28), (30) and (33), a filter clogging factor f(%), which the reduction in ease of filtering of the filter is represented by the decreasing rate in a cross sectional area of membrane pore can be calculated by Equation (3):
f=100{1−[10⁻⁹·k·τΔX·η_{w}·Q_{f}/r₀²/Aₖ/Aₘ/ΔP_{w}′]^{0.5}} Equation (3)

From Equations (29) to (31), a correction coefficient k(−) in Equation (3) can be calculated by Equation (34):
k=10⁹·r₀²·Aₖ·AₘΔP_{w0}′/τ/ΔX/η_{w0}/Q_{f0} Equation (34)
where the respective parameters represent the following:
Q_{f0}: the filtering flow rate in the priming (ml/min)
r₀: the radius of the hollow-fiber without clogging (m)
τ: a rate of curved path (−)
ΔX: membrane thickness (m)
ΔP_{w0}′: a pressure difference between the blood side end and the liquid waste side of the hollow-fiber membrane pore in the priming (mmHg)
η_{w0}: the viscosity of the priming liquid (Pa·sec)
a₀: cross sectional area of membrane pore without clogging (m²)
Aₖ: the ratio of the cross sectional area of a membrane pore to a unit area (−)
Aₘ: membrane area (m²)

Equation (3) includes a parameter of filter structure information (a radius of membrane pore of hollow-fiber without clogging, the ratio of the cross sectional area of a membrane pore to a unit area and membrane area). Thus, it is impossible to calculate a filter clogging factor using Equation (3), if filter structure information is not given. Therefore, if filter structure information is not given, a filter clogging factor f(%), which the reduction in ease of filtering of the filter is represented by the decreasing rate in a cross sectional area of membrane pore can be calculated by Equation (4) which does not include a parameter of filter structure information, by setting a correction coefficient k′(−) obtained by Equation (35):
k′=10⁻⁹·k·τ·ΔX/r₀²/Aₖ/Aₘ Equation (35)
f=100[1−(k′-η_{w}·Q_{f}/ΔP_{w}′)^{0.5}] Equation (4)

From Equations (34) and (35), a correction coefficient k′(−) in Equation (4) can be calculated by Equation (36):
k′=ΔP_{w0}′/η_{w0}/Q_{f0} Equation (36)
where the respective parameters represent the following:
Q_{b0}: a flow rate of the priming liquid that flows through the hollow-fiber in the priming (ml/min)
Q_{f0}: a filtering flow rate in the priming (ml/min)
ΔP_{b0}′: a pressure difference between both ends of the hollow-fiber in the priming (mmHg).

Furthermore, the rate of change per unit time of clogging factor f in lateral direction Δf (%/min) can be calculated by using the following Equation (37).
Δf=df/dt Equation (37)
where t represents time (min).
(Calculation of Filter Clogging Factor [s(−)] in Lateral Direction)

From the above Equations (28) and (29), obtained is a filter clogging factor s(−) which the reduction in ease of filtering of the filter is represented by a ratio of a cross sectional area inside the hollow-fiber.
S=(η_{w}*Q_{f}·ΔP_{w0}′/η_{w0}*Q_{f0}*ΔP_{w}′)^{0.5} Equation (6)
where the respective parameters represent the following:
η_{w}: viscosity of the liquid waste (Pa sec)
η_{b0}: viscosity of the liquid waste in the priming (Pa·sec)
Q_{f}: filtering flow rate (m/min)
Q_{f0}: filtering flow rate in the priming (ml/min)
ΔP_{w}′: a difference of the pressure between blood side end and liquid waste side end of the hollow-fiber membrane pore (mmHg)
ΔP_{w0}′: a difference of the pressure between blood side end and liquid waste side end of the hollow-fiber membrane pore in the priming (mmHg)

Thus, calculated is a filter clogging factor [s(−)] which the reduction in ease of filtering of the filter is represented by a ratio of a cross sectional area inside the hollow-fiber using flow rate information, measured pressure indices and biometric information (viscosity information and so on). This makes it possible to eliminate the influences of errors included in the correction coefficient (k,k′) in the calculation equation of the filter clogging factor and monitor the filter clogging more precisely irrespective of factors affecting the pressure indices (filter structure, blood purification apparatus, blood purification circuit, flow rate, biometric factor).

In this embodiment, parameters r₀, Aₖ, Aₘ, τ and ΔX are defined by the type of the filter and these parameters are collected as follows:
r₀, Aₖ, Aₘ, τ and ΔX: Manually input.
Q_{f}: Set by the blood purification apparatus and automatically input continuously in real-time.

η_{w}: Viscosity of liquid waste is measured several times a day using a viscometer and the inspection result is manually input.

ΔP_{w}′: Terms other than τΔΠ are measured by the blood purification apparatus or pressure information collection apparatus and automatically input continuously in real-time. For τΔΠ, the colloidal osmotic pressure of blood is measured by the colloidal osmotic pressure measurement several times a day and the inspection result is manually input or a blood albumin level Alb and blood globulin level Glob are measured several times a day and a value obtained through an approximate calculation using the following Equation (38) is manually input.
ΔΠ=5.54Alb+1.43Glob Equation (38)
ΔΠ: Colloidal osmotic pressure by protein (mmHg)
Alb: Blood albumin level (g/dl)
Glob: Blood globulin level (g/dl)
σ: Staverman's coefficient of restitution (proportion of solute that cannot permeate membrane) (−)
   By the way, the input method is not limited to the above-described methods.

As described above, calculating a clogging factor based on Equations (1) to (6) allows the clogging of a filter to be monitored more accurately irrespective of the presence/absence of factors affecting the pressure indices. Here, the influences of the filter on the pressure indices can be removed by considering the filter structure information (diameter of hollow-fiber, effective length of hollow-fiber, membrane area, membrane thickness, rate of hollow area, rate of curved path, diameter of membrane pore, etc.). Furthermore, the influences of the flow rate on the pressure indices can be removed by considering the flow rate information (blood flow rate, filtering flow rate, dialysis flow rate, etc.). Furthermore, the influences of biometric factors on the pressure indices are removed by integrating the measured pressure indices (Pa, Pv, Pf1, Pf2) and biometric information (viscosity (η_{b}, η_{w}), etc.).

Furthermore, errors (a difference between filter structure information of filter that is used actually and published standard values) including filter structure information or the influences of the blood purification circuit on the pressure measurements can be corrected by setting coefficients (K, k) calculated from pressure indices during priming in the clogging factor.

Furthermore, the influences of the kinds of the filter on the pressure indices or the influences of the blood purification circuits on the pressure measurements can be corrected by setting coefficients (K, k) calculated from pressure indices during priming in the clogging factor, and it is possible to calculate the clogging factor even if filter structure information is not known.

Calculating this clogging factor makes it possible to express the clogging situation of the filter with a single factor when blood purification therapy is applied to patients in various clinical conditions by calculating this clogging factor and using various filters, blood purification apparatuses and blood purification circuits with various flow rate settings, and make comparisons.

The following effects can be expected to be achieved using this clogging factor, which would be impossible to be achieved using conventional pressure indices:

1) Allows Safe Blood Purification Therapy Hardly Depending on Experiences

Use of this clogging factor allows even medical staff of little experience to simply grasp the clogging situation and also allows medical staff of rich experience to easily grasp the clogging situation when a new type of filter is used for the first time. Calculating the clogging factor in real time makes it possible to speedily take actions (increase in amount of coagulant, variation in blood flow rate) corresponding to the filter clogging.

2) Allows Collection or Exchange of Information Among Many Facilities

It is possible to evaluate the performance of and operation conditions a filter at various facilities with different blood purification apparatuses and collects information on the development of the filter and setting of optimum operation conditions.

3) Provides a Basic Step Toward Automation

When it is an aim to automate the blood purification therapy using a blood purification apparatus, if the degree of clogging can be expressed using a single index called a “clogging factor” that integrates all kinds of influencing factors, it will be possible to control the clogging more simply.

For a TMP, the above Equation (27) and the following Equations (39) to (48) can be used. Which equation should be used to calculate the TMP can be determined according to the purpose as appropriate.
TMP1=(Pa+Pv)/2−(Pf1+Pf2)/2 Equation (39)

This Equation (39) (b in FIG. 4) expresses TMP in the center of the filter.
TMP2=(Pa+Pv)/2−Pf1 Equation (40)

This equation is a clinically defined equation of a hemofiltering, hemodiafiltering or plasmapheresis apparatus. Furthermore, this Equation (40) (c in FIG. 4) indicates a difference between the pressure on the blood side in the center of the filter and filtering pressure of the blood inflow portion.
TMP3=(Pa+Pv)/2−Pf2 Equation (41)

This Equation (d in FIG. 4) is obtained by replacing the portion of measuring Pf in Equation (40) by the blood outflow portion.
TMP5=(Pa+Pv)/2−Pf1 Equation (42)

This Equation (e in FIG. 4) is a clinically defined equation of a hemofiltering, hemodiafiltering or plasmapheresis apparatus one generation ago and only represents a TMP of the blood inflow portion.
TMP2=(Pa+Pv)/2−Pf1 Equation (43)

This Equation (f in FIG. 4) is a defined equation to define a blood dialyzing apparatus. This equation also represents a difference between a pressure on the blood side of the blood outflow portion of the filter and a filtering pressure of the blood inflow portion.
TMP6=Pv−Pf2 Equation (44)

This Equation (g in FIG. 4) only represents a TMP of the blood outflow portion.
TMP7=Pa−(Pf1+Pf2)/2 Equation (45)

This Equation (h in FIG. 4) expresses a difference between a pressure on the blood side of the blood inflow portion of the filter and a filtering pressure at the center.
TMP8=Pv−(Pf1+Pf2)/2 Equation (46)
This Equation (i in FIG. 4) represents a difference between a pressure on the blood side of the blood outflow portion of the filter and a filtering pressure at the center.

In order to accurately express a filtering pressure that contributes to the passage of substances through membrane pores of the filter (effective filtering pressure), it is desirable to further calculate the above-described TMP in combination with the colloidal osmotic pressure information of the blood. Especially, Equation (39) (b in FIG. 4), Equation (42) (e in FIG. 4) and Equation (44) (g in FIG. 4) can be combined with the colloidal osmotic pressure of blood to obtain Equation (27) (j in FIG. 4), Equation (47) (k in FIG. 4) and Equation (48) (l in FIG. 4), respectively and it is thereby possible to express the filtering pressure that contributes to actual movement of substances (effective filtering pressure).
TMP9=(Pa+Pv)/2−(Pf1+Pf2)/2−σΔΠ Equation (27)
ΔΠ: Colloidal osmotic pressure by protein (mmHg)
σ: Staverman's coefficient of restitution (proportion of solute that cannot permeate membrane (−))

This equation (j in FIG. 4) represents an effective filtering pressure at the center of the filter. The pressure that contributes to the actual passage at the center of the filter (effective filtering pressure) is calculated from this equation considering an osmotic pressure (ΔΠ) by the membrane impermeable substances that exist on the blood side.
TMP10=Pa−Pf1−σΔΠ Equation (47)

This equation (k in FIG. 4) represents an effective filtering pressure at the blood inflow portion of the filter. The pressure that contributes to the actual passage at the blood inflow portion (effective filtering pressure) is calculated from this equation considering an osmotic pressure (σΔΠ) by the membrane impermeable substances that exist on the blood side.
TMP11=Pv−Pf2−σΔΠ Equation (48)

This equation (1 in FIG. 4) represents an effective filtering pressure at the blood outflow portion of the filter. The pressure that contributes to the actual passage at the blood outflow portion (effective filtering pressure) is calculated from this equation considering an osmotic pressure (σΔΠ) by the membrane impermeable substances that exist on the blood side.

In Equation (27), an average between the value obtained from Equation (47) and the value obtained from Equation (48) is calculated. This value is a typical TMP in the filter. Therefore, a clogging factor in lateral direction is calculated by substituting this value into Equation (3), (4) or (6). By the way, the value is not limited to the average between the value obtained from Equation (47) and the value obtained from Equation (48), but it is also possible to use a TMP value obtained by other methods if it is at least a typical value of TMP of the filter. Thus, calculating the blood colloidal osmotic pressure information in combination makes it possible to grasp clogging in lateral direction of the filter precisely.

When a hemodialysis or hemodiafiltering is in progress, filtering (forward filtering) is performed from the blood side to the liquid waste (dialyzing fluid) side. In this case, back filtration whereby the effective difference of the pressure is inverted near the blood outflow portion of the filter may take place (shaded area in FIG. 5). This back filtration is more likely to occur in a filter with higher solute permeability. With the recent increase in the use of a filter with high solute permeability, which is advantageous in eliminating low molecular weight protein, back filtration is more likely to occur.

Once back filtration takes place, harmful substances such as endotoxin contained in a dialyzing fluid are mixed with the blood, provoking a danger of doing harm to the patient such as high fever. However, back filtration can also be used to prevent clogging of membranes or promote exchange of substances and new types of hemodiafiltering (push and pull hemodiafiltering, hemodiafiltering using a diaphragm dialyzer, semi-nephron hemodiafiltering, super flux hemodiafiltering, etc.), which positively take advantage of this back filtration, are also being practiced.

Adopting a TMP using the above Equations (27), (47) and (48) indicating effective filtering pressures makes it possible to precisely grasp and appropriately handle clogging in lateral direction including back filtration.

As shown in this embodiment, by measuring at least two pressures (4 pressures in this embodiment) selected from a group consisting of a pressure in the blood inflow portion, pressure in the blood outflow portion, filtering pressure in the blood inflow portion and filtering pressure in the blood outflow portion, it is possible to grasp the above-described back filtration noninvasively, continuously, in real-time, precisely and specifically. This makes it possible to adjust the amount of dosage of an anticoagulant appropriately and change the setting of the flow rate of the blood.

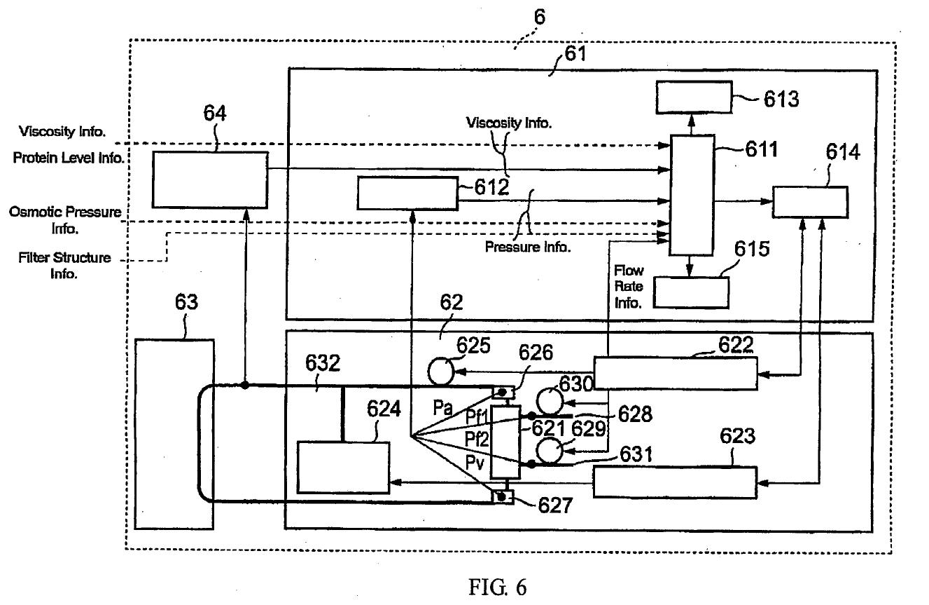

Then, a bed-side system that implements the method according to the present invention will be explained. The bed-side system 6 shown in FIG. 6 is composed in such a way that the flow rate of blood and amount of dosage of medicine can be adjusted based on the information from a continuous hematoclit monitor 64 and information from a filter monitoring apparatus 61.

This bed-side system 6 is mainly constructed of the filter monitoring apparatus 61 that monitors clogging of a filter 621 for blood purification, the continuous hematoclit monitor 64 that stores, controls and displays various kinds of information from a patient 63 and a blood purification apparatus 62 that performs blood purification processing based on the information from the filter monitoring apparatus 61, adjusts the amount of medicine administered to the patient and adjusts the flow rate of the blood.

As shown in FIG. 6, the filter monitoring apparatus 61 is mainly constructed of a pressure measurement section 612 that measures a pressure at the filter 621, a calculation section 611 that calculates a filter clogging factor from the information from the continuous hematoclit monitor 64, pressure information from the pressure measurement section 612, flow rate information from the blood purification apparatus, filter structure information and other information (viscosity information, protein concentration information, colloidal osmotic pressure information, filter structure information), a memory 613 that stores various kinds of information used for the filter clogging factor and calculation, and a display section 615 that displays the various kinds of information used for the filter clogging factor and calculation.

As shown in FIG. 6, the blood purification apparatus 62 is mainly constructed of the filter 621, a blood inflow portion-side drip-chamber 626 provided before the filter 621, a blood outflow portion-side drip-chamber 627 provided after the filter 621, a rotary pump 625 provided on a blood circulation path 632 before the blood inflow portion-side drip-chamber 626, rotary pumps 630 and 629 that adjust the flow rate of liquid waste provided for tubes 628 and 631 mounted on a coupler of the filter 621, a flow rate control section 622 that controls the flow rate of the blood of the rotary pump 625 provided on the blood circulation path 632 based on the information on the filter monitoring apparatus 61, a medicine dosage section 624 that doses medicine such as an anticoagulant into the blood circulation path 632, and a medicine dosage amount control section 623 that controls the amount of medicine dosed into the blood circulation path 632 based on the information on the filter monitoring apparatus 61.

The operation of the bed-side system in the above-described configuration will be explained.

The blood circulates from the patient 63 along the blood circulation path 632 and returns to the patient. 63 through the filter 621 mounted on the blood purification apparatus 62. At the blood purification apparatus 62, a pressure Pa at the blood inflow portion-side drip-chamber 626, pressure Pv at the blood outflow portion-side drip-chamber 627, pressure Pf1 at the coupler on the blood inflow portion side of the filter 621, and pressure Pf2 at the coupler on the blood outflow portion side of the filter 621 are measured by the pressure measurement section 612 of the filter monitoring apparatus 61. Here, the pressure Pa at the blood inflow portion-side drip-chamber 626 corresponds to the pressure in the filter blood inflow section, the pressure Pv at the blood outflow portion-side drip-chamber 627 corresponds to the pressure of the filter blood outflow section, the pressure Pf1 at the coupler on the blood inflow portion side of the filter 621 corresponds to the filtering pressure of the filter blood inflow portion and the pressure Pf2 at the coupler on the blood outflow portion side of the filter 621 corresponds to the filtering pressure of the filter blood outflow portion.

These pressures are output from the pressure measurement section 612 to the calculation section 611. The calculation section 611 calculates a filter clogging factor based on the pressure information from the pressure measurement section 612, patient information from the continuous hematoclit monitor 64, viscosity information from the outside, protein concentration information, colloidal osmotic pressure information, filter structure information and flow rate information from the blood purification apparatus 62. The filter clogging factor in vertical direction is calculated from the above-described Equation (1), (2) or (5) using at least two of blood viscosity information calculated using an Ht value from the continuous hematoclit monitor 64 and a TP value obtained from a clinical inspection, filter structure information, the pressure information from the pressure measurement section 612 and flow rate information obtained from the blood purification apparatus 62. The Ht value that determines a viscosity of blood can be collected continuously using the continuous hematoclit monitor. On the other hand, the filter clogging factor in lateral direction is calculated from the above-described Equation (3), (4) or (6) using at least two of the liquid waste viscosity information obtained from a clinical inspection, TMP calculated using the blood colloidal osmotic pressure information obtained from the pressure measurement section 612 and a clinical inspection, filter structure information and the flow rate information obtained from the blood purification apparatus 62.

The filter clogging factor calculated from the calculation section 611 is output to the control section 614. The control section 614 controls the flow rate control section 622 and the medicine dosage amount control section 623 of the blood purification apparatus 62. The flow rate control section 622 controls the flow rate of the blood that circulates inside the circulation path 632 based on the filter clogging factor. For example, the flow rate control section 622 sets an optimal blood flow rate based on a table that associates a filter clogging factor with a blood flow rate and outputs the flow rate information to the rotary pump 625. The rotary pump 625 adjusts the flow rate of the blood based on the flow rate information from the flow rate control section 622.

Furthermore, the flow rate control section 622 controls the flow rate of liquid waste that passes through the tubes 628 and 631 of the filter 621 based on the filter clogging factor. For example, the flow rate control section 622 sets an optimal liquid waste flow rate based on a table that associates a filter clogging factor with a liquid waste flow rate and outputs the flow rate information to the rotary pumps 630 and 629.

The rotary pumps 630 and 629 adjust the flow rate of liquid waste based on the flow rate information from the flow rate control section 622. At this time, the flow rate control section 622 can control the rotary pumps 630 and 629 equally or control them individually according to the clogging situation of the filter 621 (can be determined using TMPs 9 to 11).

The medicine dosage amount control section 623 controls the amount of medicine to be dosed into the circulation path 632 based on the filter clogging factor. For example, the medicine dosage amount control section 623 sets an optimal amount of medicine dosage based on a table that associates filter clogging information with an amount of medicine dosage and outputs the medicine dosage amount information to the medicine dosage section 624. The medicine dosage section 624 adjusts the amount of medicine dosage based on the medicine dosage amount information from the medicine dosage amount control section 623 and doses the adjusted amount of medicine dosage into the circulation path 632.

More specifically, when the filter clogging factor in vertical direction F increases and/or the filter clogging factor in vertical direction S decreases, the medicine dosage amount control section 623 makes a setting so as to increase the amount of dosage of an anticoagulant and controls the medicine dosage section 624 so that this amount of the anticoagulant is dosed into the circulation path 632. Furthermore, the flow rate control section 622 makes a setting so as to increase the blood flow rate and controls the rotary pump 625 so that the blood is circulated at this flow rate. This can prevent the progress of filter clogging and extend the time until the filter is clogged. Furthermore, this can also prevent the blood inside the filter, when the blood in the circuit is completely returned to the patient to terminate blood purification, from remaining (residual blood) or prevent blood loss because of the inability to return the blood in the circuit to the patient due to drastic clogging (inability to recover blood).

When the filter clogging factor f in lateral direction increases and/or the filter clogging factor s in lateral direction decreases, the medicine dosage amount control section 623 makes a setting so as to increase the amount of dosage of an anticoagulant and controls the medicine dosage section 624 so that this amount of the anticoagulant is dosed into the circulation path 632. Furthermore, the flow rate control section 622 makes a setting so as to decrease the flow rate of blood and controls the rotary pumps 630 and 629 so that the liquid waste is filtered at this flow rate. This can reduce the filtering performance per unit time and extend the time until the filter is clogged. Here, a case where control is performed to reduce the flow rate of liquid waste is explained, but it is also possible to perform control to increase the flow rate of liquid waste according to the situation.

Thus, the bed-side system according to this embodiment can perform calculation of the filter clogging factors and monitoring of filter clogging, etc., according to this embodiment at the bed side in real-time. This bed-side system can also store information collected or analyzed at the bed side and use the information to adjust the flow rate of blood or liquid waste or the amount of medicine dosage, etc.

The configurations of the bed-side system and the filter monitoring apparatus are not limited to the configurations shown in FIG. 6. That is, it is possible to calculate the filter clogging factor from the above Equations (1) to (6) and change the configuration of the apparatus based on the information in various ways within a range in which blood purification is controllable.

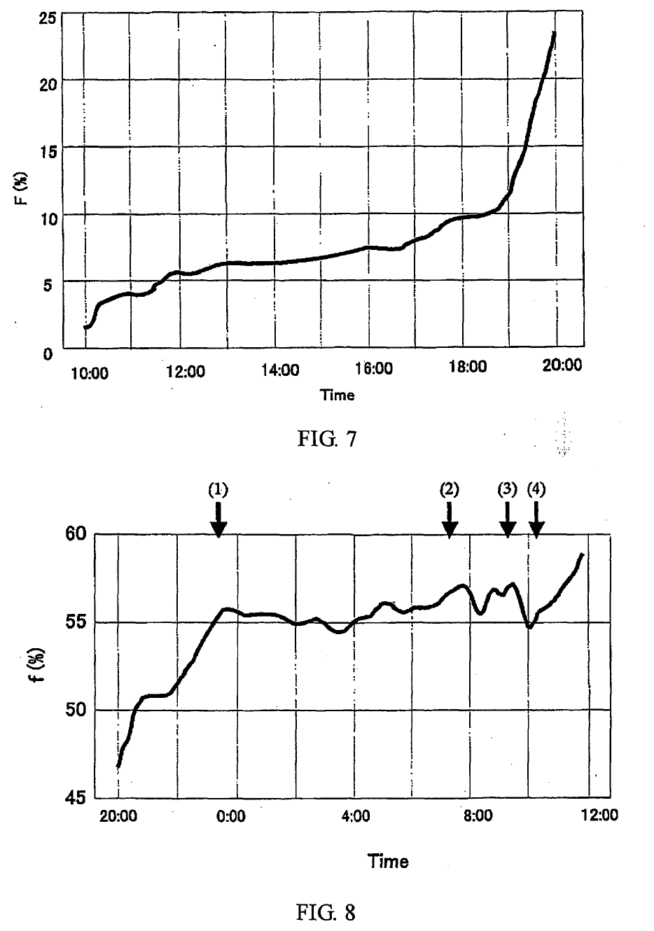

Then, practical examples that have been conducted to verify the effects of the present invention will be explained. FIG. 7 illustrates a variation of the clogging factor (F) in the vertical direction when sustained blood filtering was performed. Here, a blood purification apparatus KM-8600P (product name, manufactured by Kuraray Medical Co., Ltd.), blood purification circuit KPD-8610 (product name, manufactured by Kuraray Medical Co., Ltd.) and hemofilter APF-06S (product name, manufactured by Asahi Medical Co., Ltd.) were used. F (%) was calculated according to Equation (2) when sustained blood filtering was performed.

As is apparent from FIG. 7, the clogging in the vertical direction that progress gradually starts to accelerate drastically around 19:00. Thus, monitoring the variation of the clogging factor (F) makes it possible to keep track of the progress of the clogging in the vertical direction.

FIG. 8 illustrates a variation of the clogging factor (f) in the horizontal direction when sustained blood filtering was performed. Here, a blood purification apparatus KM-8600P (product name, manufactured by Kuraray Medical Co., Ltd.), blood purification circuit KPD-8610 (product name, manufactured by Kuraray Medical Co., Ltd.) and hemofilter APF-06S (product name, manufactured by Asahi Medical Co., Ltd.) were used f (%) was calculated according to Equation (4) when sustained blood filtering was performed.

As is apparent from FIG. 8, because the clogging factor f increased, dosage of an anticoagulant (nafamostat mesylate) was increased temporarily from 20 mg/hr to 25 mg/hr at the point (1) in the figure and as a result, the increase of f was suppressed. Then, an increase of f was observed again and so dosage of the anticoagulant (nafamostat mesylate) was increased from 20 mg/hr to 25 mg/hr at the point (2) and a new anticoagulant (low molecular weight heparin) was dosed at a rate of 100 U/hr at the point (3). This caused the clogging factor f to show a declination. However, since the amount of urine started to decrease with the deterioration of the condition of the whole body, the flow rate of filtering was increased at the point (4) to increase the amount of harmful substances removed from the body, and as a result drastic progress of clogging in the horizontal direction of the filter (drastic increase of f) was observed.

Monitoring the clogging factor in the horizontal direction in this way makes it possible to adjust dosage of the anticoagulant appropriately.

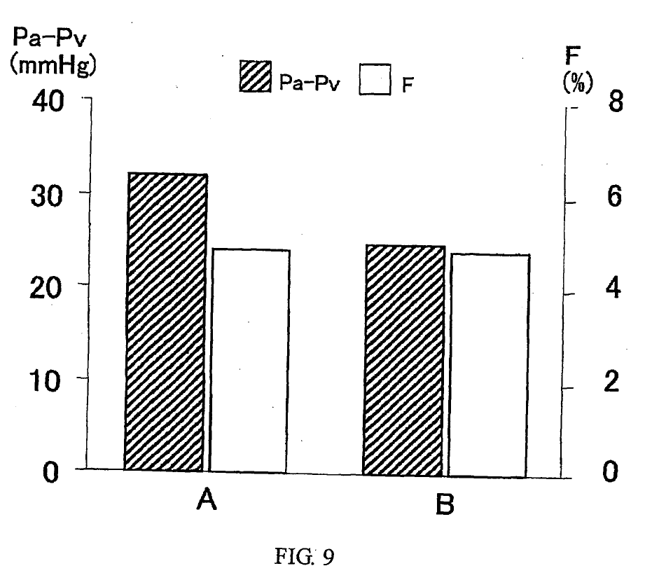

FIG. 9 illustrates a variation of a pressure index Pa−Pv and a variation of the clogging factor (F) in the vertical direction caused by a variation in the blood flow rate. Here, a blood purification apparatus KM-8600P (product name, manufactured by Kuraray Medical Co., Ltd.), blood purification circuit KPD-8610 (product name, manufactured by Kuraray Medical Co., Ltd.) and hemofilter APF-06S (product name, manufactured by Asahi Medical Co., Ltd.) were used. FIG. 9 shows the pressure index Pa−Pv in a stabilization period (A) with a blood flow rate of 100 m/min and filtering flow rate of 15 ml/min after sustained blood filtering is started and the clogging factor F (%) in the vertical direction calculated using Equation (2), and the pressure index Pa−Pv immediately after only the blood flow rate is reduced to 80 m/min (B) and the clogging factor F (%) in the vertical direction calculated using Equation (2).

The degrees of filter clogging immediately after the blood flow rate is changed (B) and immediately before the blood flow rate is changed (A) are considered to be the same. As is apparent from FIG. 9, even if the blood flow rate is reduced, the clogging factor F does not change but the pressure index Pa−Pv decreases. Thus, when the flow rate changes, it is difficult to monitor the filter clogging accurately with the pressure index alone and it is appreciated that the clogging factor of the present invention is appropriate as the parameter to monitor the clogging condition.

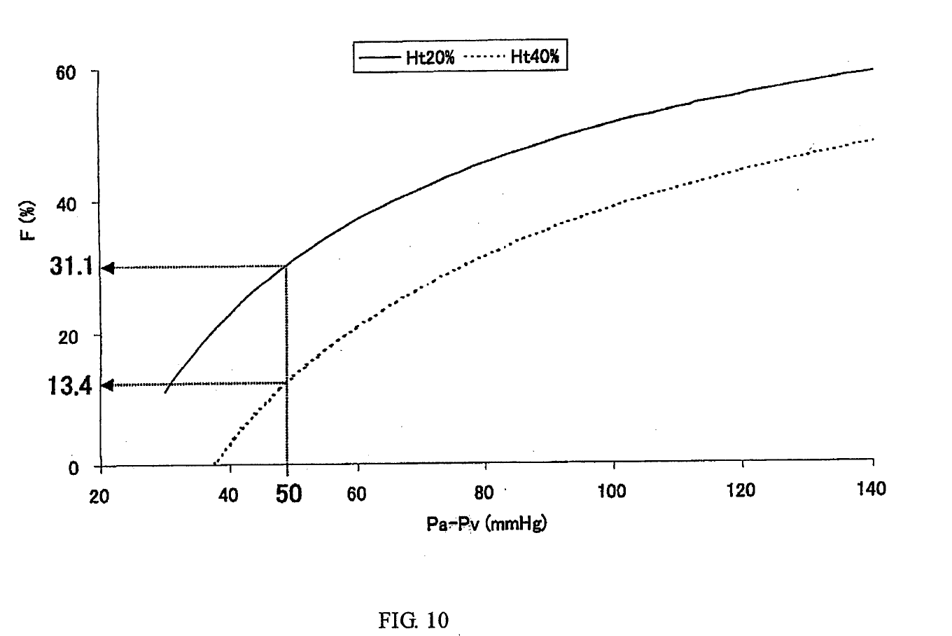

FIG. 10 shows a simulation curve indicating a relationship between the pressure index Pa−Pv and clogging factor F (%) in the vertical direction calculated from Equation (2) when sustained blood filtering was applied to a patient with a blood flow rate of 100 ml/min, filtering flow rate of 15 ml/min and total serum protein concentration of 7.0 g/dl. A blood purification apparatus ACH-10 (product name, manufactured by Asahi Medical Co., Ltd.), blood purification circuit CHF-400N (product name, manufactured by Asahi Medical Co., Ltd.) and hemofilter APF-06S (product name, manufactured by Asahi Medical Co., Ltd.) were used. As is apparent from FIG. 10, when the pressure index Pa−Pv is 50 mmHg, F is 31.1% when a hematocrit value is 20%, while F is 13.4% when the hematocrit value is 40%.

Thus, it is appreciated that even if only the pressure index Pa−Pv is monitored, when biometric information (factors affecting blood viscosity) changes, it is not possible to evaluate the filter clogging accurately. From FIG. 9 and FIG. 10, it is appreciated that the pressure index Pa−Pv is not sufficient as the parameter for monitoring the filter clogging factor.

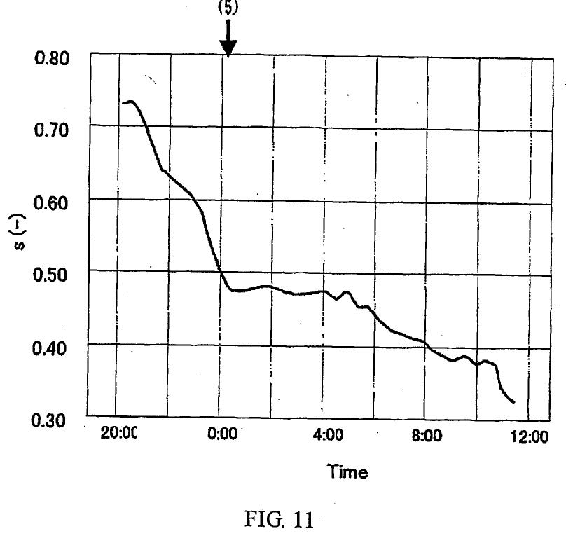

FIG. 11 illustrates a variation of the clogging factor (s) in the horizontal direction when sustained blood filtering was performed. Here, a blood purification apparatus KM-8600P (product name, manufactured by Kuraray Medical Co., Ltd.), blood purification circuit KPD-8610 (product name, manufactured by Kuraray Medical Co., Ltd.) and hemofilter APF-06S (product name, manufactured by Asahi Medical Co., Ltd.) were used.

FIG. 11 shows a variation of the clogging factor s (%) in the horizontal direction calculated using Equation (6) when sustained blood filtering was performed. At the point (5) in the figure, a 10 mg anticoagulant (nafamostat mesylate) was dosed into the blood purification circuit in one shot and the sustained dosage was increased from 20 mg/hr to 25 mg/hr, and as a result the s value was suppressed approximately 4 hours later.

Thus, monitoring the clogging factor in the horizontal direction makes it possible to adjust dosage of the anticoagulant, etc., appropriately.

From the above-described practical examples, it is appreciated that the clogging factors F, f and s of the present invention are appropriate as parameters to monitor the clogging situation of the filter.

Thus, the method according to this embodiment can discover clogging of a filter in an early stage, adjust dosage of the anticoagulant appropriately without overdosage and change the setting of the flow rate of blood to prevent the progress of clogging of the filter. Furthermore, it is also possible to predict the time during which blood purification can be executed (completion timing), which allows medical staff to prepare for terminating blood purification with a sufficient time. Furthermore, it can also prevent blood loss caused by the blood remaining in the filter (residual blood) at the end of blood purification. It also reduces the danger of blood cells being suctioned by a strong negative pressure, causing destruction of blood cells (hemolysis, etc.). It further allows more effective operating conditions to be set considering the reduction in substance removing ability (clearance) due to filter clogging.

By controlling back filtration, it is also possible to set the flow rate considering back filtration of each filter.

Thus, by preventing filter clogging and controlling back filtration, it is possible to perform blood purification more safely and economically.

Furthermore, it is also possible to evaluate how clogging occurs in each filter and how back filtration occurs, and use these evaluation results for the development of a filter in which clogging hardly occurs or the development of a filter with controlled back filtration.

The present invention is not limited to the above-described embodiments, but can be implemented modified in various ways. For example, the numerical values and materials in the above-described embodiments are presented for illustrative purposes and not limitative, and can be implemented modified in various ways.

The present invention is applicable to an evaluation of clogging for tubular-like structures, for example water purification apparatus, transport tube for a liquid medicine, a water pipe, ink pipe, ink nozzle, or spraying tube for a liquid medicine.

As explained above, the present invention measures at least two pressures selected from the group consisting of a pressure in the blood inflow portion, a pressure in the blood outflow portion, a filtering pressure in the blood inflow portion, and a filtering pressure in the blood outflow portion and calculates a filter clogging factor in vertical direction and lateral direction by using at least two of the measured pressures, flow rate information (conditions during operation), biometric information (viscosity information), a correction coefficient calculated from pressure indices in the priming, structure information. Thereby, it is possible to discover filter clogging in vertical direction and/or lateral direction in an early stage, appropriately adjust dosage of an anticoagulant without overdosage, change a blood flow rate setting and prevent the progress of filter clogging.

This application is based on the Japanese Patent Application No. 2002-187949 filed on Jun. 27, 2002, entire content of which is expressly incorporated by reference herein.

1. Method for calculating a clogging factor of a filter composed of hollow-fiber membrane, which has a blood inflow portion and a blood outflow portion, for filtering a blood by passing said blood, said method comprising the steps of:
measuring at least two pressure selected from the group consisting of a pressure in said blood inflow portion, a pressure in said blood outflow portion, a filtering pressure in said blood inflow portion, and a filtering pressure in said blood outflow portion; and
calculating a filter clogging factor indicating the reduction in flowing ease of the blood in said filter and/or a filter clogging factor indicating the reduction in ease of filtering of said filter, by using the measured pressure.

2. Method for calculating a clogging factor of a filter according to claim 1, wherein a filter clogging factor indicating the reduction in flowing ease of the blood in said filter is calculated by using a viscosity of blood.

3. Method for calculating a clogging factor of a filter according to claim 1, wherein a filter clogging factor indicating the reduction in ease of filtering of said filter is calculated by using a viscosity of liquid waste.

4. Method for calculating a clogging factor of a filter according to claim 1, wherein a filter clogging factor indicating the reduction in flowing ease of the blood in said filter is calculated by using structure information and/or flow rate information of said filter.

5. Method for calculating a clogging factor of a filter according to claim 1, wherein a filter clogging factor indicating the reduction in ease of filtering of said filter is calculated by using structure information and/or flow rate information of said filter.

6. Method for calculating a clogging factor of a filter according to claim 2, wherein a filter clogging factor [F(%)], which the reduction in flowing ease of the blood in said filter is represented by the decreasing rate in a cross sectional area inside said hollow-fiber, is calculated by using the Equation (1):
F=100{1−[10−9·K·l·η_{b}·(Q_{b}−Q_{f}/2)/N/ΔP_{b}′/π]^{0.5}/R₀²} Equation (1)
where K represents a correction coefficient (−), 1b represents viscosity (Pa·sec) of the blood, Q_{b} represents flow rate (ml/min) of the blood flowing into the filter, Q_{f} represents filtering flow rate (ml/min), N represents the number of hollow-fibers (−), ΔP_{b}′ represents a difference (mmHg) of the pressure between both ends of the hollow-fiber, I represents an effective length (m) of the hollow-fiber, and R₀ represents the radius (m) inside the hollow-fiber that the clogging does not occur.

7. Method for calculating a clogging factor of a filter according to claim 2, wherein a filter clogging factor [F(%)] which the reduction in flowing ease of the blood in said filter is represented by the decreasing rate in a cross sectional area inside said hollow-fiber is calculated by using the Equation (2):
F=100{1−[K′·η_{b}·(Q_{b}−Q_{f}/2)/ΔP_{b}′]0.5} Equation (2)
where K′ represents a correction coefficient (−), η_{b} represents viscosity (Pa·sec) of the blood, Q_{b} represents flow rate (ml/min) of the blood flowing into the filter, Q_{f} represents filtering flow rate (ml/min), and ΔP_{b}′ represents a difference (mmHg) of the pressure between both ends of the hollow-fiber.

8. Method for calculating a clogging factor of a filter according to claim 1, a filter clogging factor indicating the reduction in flowing ease of the blood in said filter is calculated in real-time.

9. Method for calculating a clogging factor of a filter according to claim 3, wherein a filter clogging factor [f(%)], which the reduction in ease of filtering of said filter is represented by the decreasing rate in a cross sectional area of pore of said hollow-fiber, is calculated by using the Equation (3):
f=100[1−(10⁻⁹·k·τ·ΔX·η_{w}·Q_{f}/r₀/Aₖ/Aₘ/ΔP_{w}′)^{0.5}] Equation (3)
where k represents a correction coefficient (−), τrepresents a rate of curved path, ΔX represents a thickness of a membrane, 11w represents a viscosity of liquid waste passing a filter (Pa·sec), Q_{f} represents filtering rate (ml/min), r₀ represents the radius (m) of a hollow-fiber membrane pore that the clogging does not occur, ΔP_{w}′ represents a difference of the pressure between the blood side end and the liquid waste side end in the membrane pore of the filter (mmHg), Aₖ represents a proportion of a cross sectional area of the membrane pore to a unit area of the membrane in the filter, and Aₘ represents an area (m) of the membrane in the filter.

10. Method for calculating a clogging factor of a filter according to claim 3, wherein a filter clogging factor [f(%)], which the reduction in ease of filtering of said filter is represented by the decreasing rate in a cross sectional area of pore of said hollow-fiber, is calculated by using the Equation (4):
f=100[1−(k′·η_{w}·Q_{f}/ΔP_{w}′)^{0.5}] Equation (4)
where k′ represents a correction coefficient (−), η_{w} represents a viscosity of liquid waste passing a filter (Pa·sec), Q_{f} represents filtering rate (ml/min), r represents the radius (m) of a hollow-fiber membrane pore that the clogging does not occur, and ΔP_{w}′ represents a difference of the pressure between the blood side end and the liquid waste side end in the membrane pore of the filter (mmHg).

11. Method for calculating a clogging factor of a filter according to claim 1, wherein, a filter clogging factor indicating the reduction in ease of filtering of said filter is calculated in real-time.

12. Method for calculating a clogging factor of a filter according to claim 1, wherein a filter clogging factor [S(−)] which the reduction in flowing ease of the blood in said filter is represented by the decreasing rate in a cross sectional area inside said hollow-fiber is calculated by using the Equation (5):
S=[η_{b}·(Q_{b}-Q_{f}/2)·ΔP_{b0}′/η_{b0}/(Q_{b0}−Q_{f0}/2)/ΔP_{b}]^{0.5} Equation (5)
wherein η_{b} represents viscosity (Pa·sec) of the blood flowing in the hollow-fiber, η_{b0} represents viscosity (Pa·sec) of the priming liquid in the priming, Q_{b} represents flow rate (ml/min) of the blood flowing into the filter, Q_{b0} represents flow rate (ml/min) of the priming liquid flowing into the filter in the priming, Q_{f} represents filtering flow rate (mmin), Q_{f0} represents filtering flow rate (mmin) in the priming, ΔP_{b}′ represents a difference (mmHg) (Pa−Pv) of the pressure between both ends of the hollow-fiber, and ΔP_{b0}′ represents a difference (mmHg) of the pressure between both ends of the hollow-fiber in the priming.

13. Method for calculating a clogging factor of a filter according to claim 1, wherein a filter clogging factor [s(−)] which the reduction in ease of filtering of said filter is represented by the decreasing rate in a cross sectional area of membrane pore of said hollow-fiber is calculated by using the Equation (6):
s=(η_{w}·Q_{f}·ΔP_{w0}′/η_{w0}/Q_{f0}/ΔP_{w}′)^{0.5} Equation (6)
wherein η_{w}, represents viscosity (Pa·sec) of the liquid waste, η_{b0} represents viscosity (Pa·sec) of the liquid waste in the priming, Q_{f} represents filtering flow rate (mmin), Q_{f0} represents filtering flow rate (ml/min) in the priming, ΔP_{w}′ represents a difference (mmhg) of the pressure between blood side end and liquid waste side end of the hollow-fiber membrane pore, Δ_{Pw}0′ represents a difference (mmHg) of the pressure between blood side end and liquid waste side end of the hollow-fiber membrane pore in the priming, and s represents a ratio of cross sectional areas in the hollow-fiber membrane pore of the filter.

14. Method for calculating a clogging factor of a filter according to claim 1, wherein, an average of ΔP_{w}′ in said blood inflow portion and ΔP_{w}′ in said blood outflow portion is used as ΔP_{w}′.

15. Method for monitoring a clogging of a filter comprising the steps of:
calculating a clogging factor of a filter by using a method for calculating a clogging factor of a filter according to any one of claim 1; and
monitoring a clogging of a filter on the basis of the clogging factor of a filter.

16. Apparatus of monitoring a clogging of a filter comprising:
means for calculating a clogging factor of a filter by using a method for calculating a clogging factor of a filter according to any one of claim 1; and
means for monitoring a clogging of a filter on the basis of the clogging factor of a filter.

17. Bed-side system comprising apparatus of monitoring a clogging of a filter according to claim 16.
